(19) United States
(12) Patent Application Publication
(10) Pub. No.: US 2025/0195402 A1
(43) Pub. Date: Jun. 19, 2025

(54) MELALEUCA ALTERNIFOLIA EXTRACT AND COSMETIC USES THEREOF

(71) Applicant: LUCAS MEYER COSMETICS, Massy (FR)

(72) Inventors: JOAN ATTIA, LAVERNOSE LACASSE (FR); ROMAIN DUROUX, COUZIEX (FR)

(21) Appl. No.: 18/849,650

(22) PCT Filed: Mar. 23, 2023
(86) PCT No.: PCT/IB2023/052867

§ 371 Date: Sep. 23, 2024

(30) Foreign Application Priority Data
Mar. 24, 2022 (FR) 2202650

(51) Int. Cl. A61K 8/9789; A61K 8/31; A61K 8/34; A61K 8/37; A61Q 19/08

(52) U.S. Cl. CPC A61K 8/9789; A61K 8/31; A61K 8/34; A61K 8/345; A61K 8/37; A61Q 19/08

(57) ABSTRACT

The present invention relates to the cosmetic field. In particular, the present invention relates to a particular oily extract of a Melaleuca alternifolia, to a cosmetic composition comprising the same, and to the use thereof as a cosmetic regeneration, repairing or anti-fatigue agent for healthy skin.

### TECHNICAL FIELD

The present invention relates to the cosmetic field, in particular to cosmetic agents for combating the effects of fatigue on the skin.

### TECHNOLOGICAL BACKGROUND

The skin is the first barrier protecting the body from external attacks. This organ is composed of several layers of tissue. A distinction is made between the epidermis, which is the outermost part of the skin, the dermis, a connective tissue consisting of fibroblasts and an extracellular matrix, which performs the skin cohesion and nutrition functions, and the hypodermis consisting of adipocytes.

The epidermis consists of several cell strata of keratinocytes. The germ layer of the epidermis, called the basal layer, containing in particular the skin stem cells, the spiny layer, Stratum spinosum, consisting of several layers of polygonal cells, the granular layer, Stratum granulosum, comprising one to three layers of flattened cells containing cytoplasmic inclusions, the keratohyalin granules, and, finally, the horny layer, Stratum corneum which is composed of anucleate cells rich in keratin, called corneocytes, which correspond to the terminal keratinocyte differentiation stage, are inter alia, singled out.

The outermost cells of the horny layer are continually eliminated and replaced by the cells of a lower layer, according to a process referred to as desquamation. The cell regeneration of the horny layer is based on a process of cell maturation in which the cells of the basal layer of the epidermis differentiate and gradually migrate through the various strata of the epidermis until they reach the horny layer in the form of corneocytes.

The skin is generally first to display signs of fatigue. Fatigue can be caused by many factors, such as lack of sleep, poor or unbalanced dietary health, overwork, intense activity or inadequate activity at work, or disruptions in the family sphere. Women juggling family life and work, and young working people in an urban environment are particularly at risk of chronic fatigue.

Individuals exposed to such fatigue may exhibit haggard features, a loss of radiance of the complexion or even a peaky complexion, or dark circles and bags under the eyes. In other words, their face appears tired and marked, or even unhealthy. Yet projecting the image of a healthy person may be a major preoccupation for working active individuals, notably in a competitive environment and/or in contact with clients.

Nevertheless, there currently remains a need for new cosmetic active agents for preventing or treating the visible effects of fatigue on the skin.

### SUMMARY OF THE INVENTION

One subject of the invention is the cosmetic use of an oily extract of Melaleuca alternifolia, as a regenerative, repairing or anti-fatigue cosmetic agent for healthy skin, wherein said extract comprises:
more than 20% of compounds selected from sesquiterpenes and sesquiterpenoids, and
less than 6% of terpinen-4-ol,
the percentages being expressed relative to the total content of volatile compounds present in the extract determined by gas chromatography coupled with a flame ionization detector (GC/FID).

Preferably, the percentages correspond to the percentage area of the peaks corresponding to the chemical compounds of interest relative to the total area of the peaks of the chromatogram obtained by GC/FID analysis on a non-polar column.

Preferably, said extract of Melaleuca alternifolia comprises less than 15%, preferably less than 10%, better still less than 2.0% of terpineols. In particular, the extract of Melaleuca alternifolia according to the invention may comprise:
less than 5.0%, preferably less than 2.0%, better still less than 1.0%, of terpinen-4-ol, and/or
less than 5.0%, preferably less than 2.0%, better still less than 1.5%, of alpha-terpineol.

In certain embodiments, said extract of Melaleuca alternifolia comprises more than 30%, preferably more than 40% of compounds selected from sesquiterpenes and sesquiterpenoids. In particular, said extract of Melaleuca alternifolia may comprise:
from 5% to 15% of aromadendrene,
from 10% to 25% of ledene,
from 10% to 25% of delta-cadinene,
from 1% to 5% of globulol, and
from 1% to 5% of viridifloral.

In one particular embodiment, the extract of Melaleuca alternifolia is obtained by fractional distillation of an essential oil of leaves and/or terminal branches of Melaleuca alternifolia. In certain embodiments, said extract of Melaleuca alternifolia is used to treat or prevent one or more signs of skin fatigue selected from a loss of radiance of the skin, a dull complexion, a peaky complexion, a loss of uniformity of the complexion, haggard facial features, dark circles around the eyes, bags under the eyes, puffy eyelids, a alteration in the smooth appearance of the skin, an increase in the roughness of the skin, a loss of elasticity, a loss of density, a loss of firmness, a loss of hydration, an appearance of wrinkles, an appearance of redness, and combinations thereof.

In other embodiments, said extract of Melaleuca alternifolia is used as a cosmetic agent for promoting or improving skin recovery during sleep and/or as a cosmetic agent for potentiating the natural mechanisms of skin repair and regeneration controlled by melatonin.

Said extract of Melaleuca alternifolia according to the invention may further be used to increase the total duration of sleep, and/or that of deep sleep.

Typically, said extract of Melaleuca alternifolia is present as a cosmetic active agent in a cosmetic composition, preferably a cream, a balm, a mask, a serum or a lotion.

Another subject of the invention is an extract of Melaleuca alternifolia as described above. A further subject is a precursor composition for the preparation of a cosmetic composition comprising from 0.1% to 1% by weight, preferably from 0.1% to 0.5% by weight, of an extract of Melaleuca alternifolia according to the invention, in a cosmetically acceptable carrier, preferably pentylene glycol or triheptanoin.

Another further subject is a cosmetic ingredient comprising from 0.1% to 1% by weight, preferably from 0.1% to 0.5% by weight, of an extract of Melaleuca alternifolia according to the invention, in a cosmetically acceptable carrier, preferably pentylene glycol or triheptanoin. Said cosmetic ingredient is preferably used as a a regenerative, repairing or anti-fatigue cosmetic agent for healthy skin.

Another subject of the invention is a cosmetic composition comprising the extract of Melaleuca alternifolia, the precursor composition or the cosmetic ingredient according to the invention, in combination with at least one cosmetically acceptable excipient.

The cosmetic composition according to the invention may comprise from 0.0005% to 0.01%, preferably from 0.001% to 0.005% by weight of said extract of Melaleuca alternifolia. Alternatively, the cosmetic composition according to the invention may comprise from 0.5% to 5% by weight, preferably from 1% to 3% by weight, of the cosmetic ingredient or of the precursor composition.

The cosmetic composition according to the invention may be selected from the group consisting of aqueous solutions, aqueous-alcoholic solutions, oil-in-water (O/W) emulsions or water-in-oil (W/O) emulsions or multiple (triple: W/O/W or O/W/O) emulsions, nanoemulsions, in particular O/W nanoemulsions, aqueous gels, or spherule-assisted dispersions of a fatty phase in an aqueous phase, suspensions, preferably in aqueous or aqueous-alcoholic media, liposome suspensions, powders, lotions, milks, creams, unguents, gels, foams, and ointments.

The cosmetic composition according to the invention may be a care product for treating or preventing skin fatigue, for example a serum, a cream, a mask or a balm, preferably for use at night.

A subject of the invention is a cosmetic method for preventing or treating signs of skin fatigue in an subject, said method comprising the application of a cosmetically effective amount of an extract of Melaleuca alternifolia or of a cosmetic composition according to the invention on the skin.

Finally, the subject of the invention is the use of an extract of Melaleuca alternifolia or of a cosmetic ingredient as described in the present application in the manufacture of a cosmetic composition for treating, preventing or reducing the signs of cutaneous fatigue. Each of the aspects and embodiments described herein is capable of being used together, unless this is explicitly excluded or excluded clearly from the context of the embodiment or aspect under consideration.

### FIGURES

FIG. 1 represents the modulation of various genes associated with skin repair and also the signaling pathway of melatonin in the presence of the extract according to the invention in skin explants subjected to UV stress compared to placebo explants (subjected to UV stress, in the absence of the extract according to the invention).

FIG. 2 shows the effect of skin application of the extract according to the invention on the face over the total duration of sleep.

FIG. 3 shows the effect of skin application of the extract according to the invention on the face over the duration of deep sleep.

FIGS. 4 to 10 show the results of the clinical trial from example 6.

FIG. 4 shows the transepidermal water loss (TEWL) for the group treated with the “active” cream (Extract according to the invention) and the group treated with the placebo cream (Placebo) at T12 h, D7 and D28 as a relative percentage with respect to D0.

FIG. 5 shows the skin hydration evaluated by corneometry for the group treated with the “active” cream (Extract according to the invention) and the group treated with the placebo cream (Placebo) at T12 h, D7 and D28 as a relative percentage with respect to D0.

FIG. 6 shows the antioxidant potential of the skin measured by the FRAP (Ferric Reducing Antioxidant Parameter) test using samples from the first layers of the stratum corneum taken on D0 and D28 on the cheeks using a Corneofix® adhesive patch for the group treated with the “active” cream (Extract according to the invention) and the group treated with the placebo cream (Placebo) on D28 as a relative percentage with respect to D0.

FIGS. 7, 8 and 9 show the results of experiments aimed at evaluating the smooth appearance and biomechanical properties of the skin. FIG. 7 shows the evolution of the parameter Sa (smooth appearance of the skin) at T12 h, D7 and D28 as a relative percentage with respect to D0. FIGS. 8 and 9 show the parameters R0 (firmness) and R2 (elasticity) as a relative percentage with respect to D0 for the group treated with the “active” cream (Extract according to the invention) and the group treated with the placebo cream.

FIG. 10 shows the results of the evaluation of dark circles at T12 h, D7 and D28 as a relative percentage with respect to D0 for the group treated with the “active” cream (Extract according to the invention) and the group treated with the placebo cream.

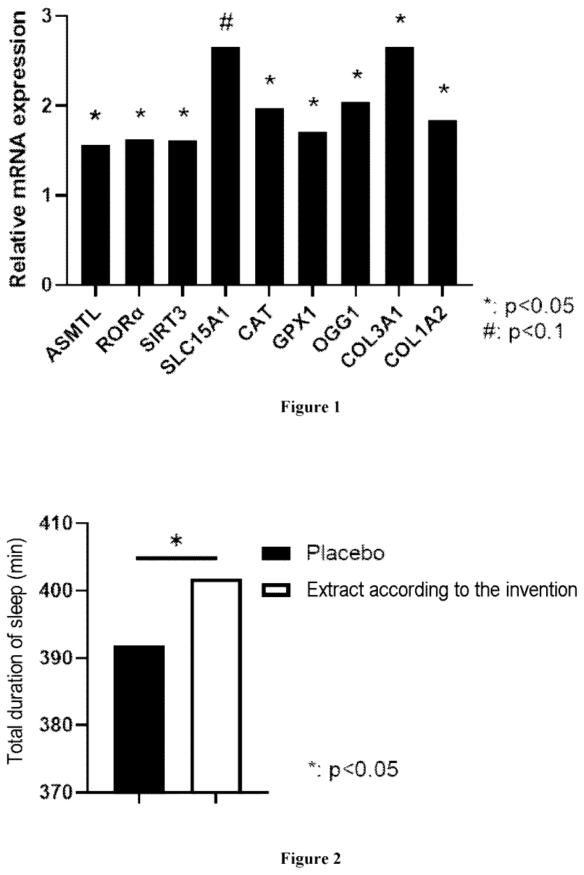

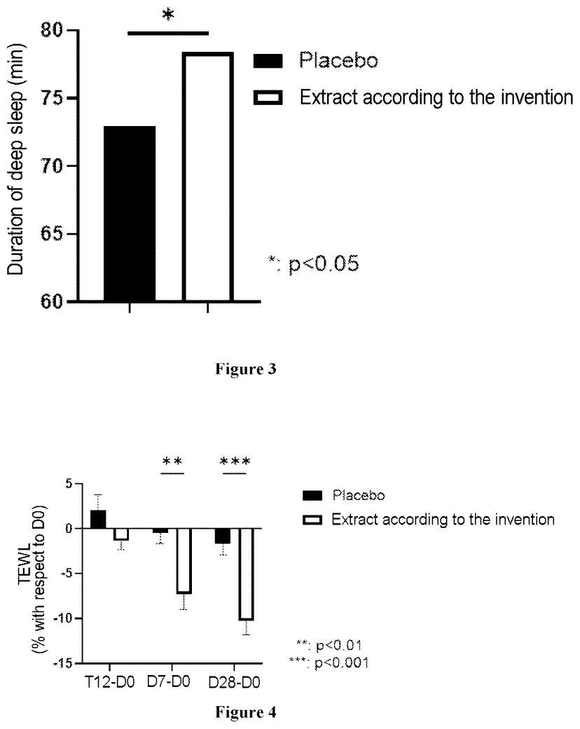

Statistical analysis: * p<0.05; ** p<0.01; *** p<0.001; **** p<0.0001.

### DETAILED DESCRIPTION

Melatonin is a hormone produced by the pineal gland (also known as epiphysis cerebri). The secretion of melatonin is inhibited in the presence of light and stimulated when it is dark. By means of the melatonin, the pineal gland informs the brain of the relative durations of the hours of darkness and light over a 24-hour period (daily cycle), but also throughout the year (seasonal cycle). Melatonin is referred to as “the sleep hormone” and plays a central role in regulating chronobiological rhythms. This hormone is produced in various tissues. The process begins with tryptophan which is converted into serotonin via various enzymes in order to eventually transform the latter into melatonin by the successive action of arylalkylamine N-acetyltransferase and hydroxyindole-O-methyltransferase. Melatonin can act on the skin by two different pathways: independently of and dependently on receptors. Melatonin is capable of carrying out a cellular antioxidant role, in particular by directly trapping the free radicals, but also indirectly by increasing the activity of antioxidant enzymes. This molecule, which has the potential to cross cell membranes, also acts to maintain and protect mitochondrial activity. Melatonin also has beneficial effects by activation of melatonin receptors. Melatonin receptors are G protein-coupled receptors which exist in two subtypes in humans, namely MTI and MT2. These two receptors create a biological rhythm which induces sleep. Melatonin receptors are expressed in the brain but also peripherally. The MTI receptor is expressed in particular in the skin. The MTI receptor is involved in the mechanisms for protecting the skin against environmental stress but also in skin aging or pigmentation.

Lastly, melatonin interacts with the nuclear receptor RORα which acts as a significant transcription factor involved in numerous biological, notably antioxidant, mechanisms.

The applicant has developed a particular extract of Melaleuca alternifolia characterized by an original composition, namely a low content of terpinen-4-ol and a high content of sesquiterpene-type and sesquiterpenoid-type compounds. This extract is derived from the fractional distillation of a conventional essential oil of Melaleuca alternifolia. Surprisingly, the applicant has demonstrated that this extract has biological activities that make it possible to act on the signaling pathway of melatonin to mimic and strengthen its activity. The applicant has notably demonstrated that the extract according to the invention is capable of activating specific genes of the signaling pathway of melatonin in skin explants subjected to UV exposure, such as ASMTL, RORα, SIRT3 and SLC15A1. Since ASMTL is an enzyme that enables melatonin synthesis and since SLC15Al is a mitochondrial melatonin transporter, the extract therefore has the ability to potentiate the effect of melatonin. Furthermore, like melatonin, the extract is capable of activating RORα and SIRT3 which play a major role against cell damage by limiting oxidative stress. Furthermore, the extract induced the expression of antioxidant enzymes such as CAT and GPX1.

The extract according to the invention was also capable of inducing genes involved in collagen synthesis (COL3A1 and COLIA2) and DNA repair (OGGI) in order to promote the natural mechanisms of skin repair and regeneration which are controlled by melatonin.

Quite surprisingly, the applicant has demonstrated that the application of the extract according to the invention at a very low concentration on the skin makes it possible to improve the quality of sleep subjects having minor sleep disorders due to their lifestyle, in particular by increasing the total duration of sleep but also the duration of deep sleep, thus promoting the natural repair mechanisms that take place in particular during this phase of sleep.

Finally, the applicant confirmed the anti-fatigue and regenerative effect of the extract of Melaleuca alternifolia according to the invention during a clinical trial carried out in healthy volunteers with disturbed sleep cycles (example 6). This clinical trial showed that the daily application of a cream comprising the extract according to the invention makes it possible to prevent and/or treat the signs of skin fatigue. The extract according to the invention notably made it possible to improve the barrier function of the skin, to improve the antioxidant power of the skin, to prevent and improve the smooth appearance and biomechanical properties of the skin and to reduce dark circles in volunteers, in a significant manner compared to the group of volunteers treated with placebo cream. The extract according to the invention also made it possible to reduce the depth of crow's feet wrinkles, which highlights an anti-wrinkle effect.

Thus, all of these results support the cosmetic use, via topical application, of the extract according to the invention as an antifatigue agent, that is to say in order to treat or prevent the cutaneous signs of fatigue, and more generally as an agent for promoting or improving the recovery of the skin during sleep, in particular by potentiating the natural skin regeneration and repair mechanisms which are controlled by melatonin.

Without wishing to be bound by any one theory, the applicant is of the opinion that these properties result from the specific composition of the extract prepared by fractional distillation of the essential oil of M. alternifolia.

To the knowledge of the applicant, the extract according to the invention and the use thereof as a cutaneous antifatigue agent have never been described or suggested in the prior art. Melaleuca alternifolia is a plant endemic to Australia that First Nations Australians traditionally use as a medicinal herb and antiseptic agent. The prior art essentially relates to the essential oil of Melaleuca alternifolia, which has a composition and uses which are different from the extract according to the invention. Specifically, the essential oil of Melaleuca alternifolia is an essential oil typically obtained by steam distillation of leaves and/or ends of branches of Melaleuca alternifolia, i.e. tea tree.

The main active component of the essential oil of M. alternifolia is terpinen-4-ol. By way of example, the essential oil of Melaleuca alternifolia as defined according to ISO 4730:2017 standard comprises, inter alia, from 35-48% of terpinen-4-ol, and also 14-28% of gamma-terpinene, 6-12% of alpha-terpinene, and 0.2-5% of alpha-terpineol.

The essential oil of M. alternifolia is traditionally used in dermatology or in medicine for its anti-acne, antibacterial and antifungal properties. The essential oil of M. alternifolia is thus used on the skin or mucous membranes to treat infections such as mycoses, skin abscesses, furuncles or else aphthae. It is also used to treat certain ENT infections or else as a repellent in aromatherapy.

One subject of the present invention is therefore said extract of Melaleuca alternifolia and the uses thereof in the cosmetics field.

a. Extract of Melaleuca Alternifolia According to the Invention

Thus, according to a first aspect, the present invention relates to extract of Melaleuca alternifolia low in terpinen-4-ol, or more generally in terpineols and in monoterpenes, and enriched in sesquiterpenes and/or sesquiterpenoids.

The extract of Melaleuca alternifolia according to the invention is liposoluble. More precisely, the extract according to the invention is an oily extract, also denoted under the term oil. Preferably, the extract of Melaleuca alternifolia according to the invention comprises:
more than 20% of compounds selected from sesquiterpenes and sesquiterpenoids, and
less than 6% of terpinen-4-ol.

Unless otherwise indicated, in the present application, the percentages of the compounds included in the extract of Melaleuca alternifolia correspond to the area percentages of the peaks corresponding to the chemical compounds of interest relative to the total area of the peaks of the chromatogram obtained by GC/FID analysis on a non-polar column, for example based on dimethylpolysiloxane, preferably carried out under the conditions described in example 1.

In certain embodiments, the extract of Melaleuca alternifolia according to the invention comprises less than 5.0%, for example less than 4.5%, 4.0% or 3.5% of terpinen-4-ol.

In certain embodiments, the extract according to the invention comprises less than 3.0%, for example less than 2.0% or 1.0% or even less than 0.5% of terpinen-4-ol.

Terpinen-4-ol is also referred to as 4-terpineol, para-menth-1-en-4-ol, or 1-isopropyl-4-methyl-3-cyclohexen-1-ol.

In certain embodiments, the extract of Melaleuca alternifolia according to the invention comprises less than 5.0%, preferably less than 2.0%, or even less than 1.5% of alpha-terpineol.

In certain embodiments, the extract of Melaleuca alternifolia according to the invention comprises, generally, less than 15.0%, preferably less than 10.0%, or even less than 8.0%, 6.0%, 5.0%, 4.0%, 3.0% or 2.0% of terpineols.

Terpineols are unsaturated monocyclic monoterpene alcohols. In the context of the present invention, the terpineols include terpinen-4-ol, alpha-terpineol, beta-terpineol, gamma-terpineol, terpinen-1-ol, and terpinen-5-ol.

In certain embodiments, the extract of Melaleuca alternifolia according to the invention comprises less than 10%, preferably less than 5% or 2%, or even less than 1% of monoterpenes. Monoterpenes are terpenes consisting strictly of two isoprene units.

In the context of the present invention, the monoterpenes include, inter alia, terpinenes such as alpha-terpinene and gamma-terpinene (or terpinolene), alpha-phellandrene, alpha-p-dimethylstyrene, alpha-pinene, beta-pinene, limonene, sabinene, para-cymene, alpha-thujene, or myrcene.

In particular, the extract of Melaleuca alternifolia according to the invention may comprise less than 5%, preferably less than 2% or even less than 1% or 0.5% of terpinenes.

In certain embodiments, the extract of Melaleuca alternifolia according to the invention comprises less than 10%, preferably less than 5% or 2%, or even less than 1% of monoterpenoids other than terpineols, such as eucalyptol, camphor, or trans-piperitol.

In certain embodiments, the extract of Melaleuca alternifolia according to the invention comprises more than 30% or 40%, or even more than 50%, 60% or 70% of compounds selected from sesquiterpenes and sesquiterpenoids.

Sesquiterpenes are terpenes consisting strictly of three isoprene units.

In the context of the present invention, the sesquiterpenes include, inter alia, aromadendrene, ledene (or “viridiflorene”), delta-cadinene, beta-caryophyllene, alloaromadendrene, valencene, beta-selinene, bicycloelemene, alpha-cubebene, beta-guaiene, alpha-copaene, alpha-gurjunene, beta-ylangene, alpha-humulene, or beta-cedrene, beta-maaliene, gamma-murolene, gamma-curcumene, gamma-cadinene, or cadina-1,4-diene.

Sesquiterpenoids are derivatives of sesquiterpenes, generally formed by synthetic or biological modification of sesquiterpenes (typically, by oxidation and/or rearrangement).

In particular, sesquiterpenoids comprise additional functional groups compared to the sesquiterpenes, notably an oxygen (for example an ester, alcohol, ketone or else etheroxide function) and/or fewer alkyl groups (for example one less methyl).

In the context of the present invention, the sesquiterpenoids include, inter alia, calamenene, alpha-calacorene, beta-calacorene, delta-cadinol, elema-1,11-dien-15-al, globulol, viridifloral, cadin-4-en-10-ol, ledol, rosifoliol, or spathulenol.

The extract according to the invention may notably comprise at least 20% (e.g. at least 25%) of the following sesquiterpene and sesquiterpenoid compounds: aromadendrene, ledene, delta-cadinene, globulol and viridifloral.

In one particular embodiment, said extract of Melaleuca alternifolia according to the invention comprises:
from 5% to 15% of aromadendrene,
from 10% to 25% of ledene, and
from 10% to 25% of delta-cadinene.

In one more particular embodiment, said extract of Melaleuca alternifolia according to the invention comprises:
from 5% to 15% of aromadendrene,
from 10% to 25% of ledene,
from 10% to 25% of delta-cadinene,
from 1.0% to 5.0% of globulol, and
from 1.0% to 5.0% of viridifloral.

In another additional embodiment, the extract according to the invention comprises:
less than 6% (for example less than 5%, 4.5%, 4.0%, 3.5%, 3.0%, 2.0%, 1%, 0.5%) of terpinen-4-ol,
less than 5%, (for example less than 2.0% or 1.5%) of alpha-terpineol,
less than 15.0% (for example less than 10.0%, 8.0%, 6.0%, 5.0%, 4.0%, 3.0% or 2.0%) of terpineols,
less than 10% (for example less than 5%, 2%, or 1%) of monoterpenes,
less than 10% (for example less than 5%, 2%, or 1%) of monoterpenoids other than terpineols,
at least 25% (for example more than 30%, 40%, 50%, 60%, or 70%) of compounds selected from sesquiterpenes and sesquiterpenoids.

Preferably, the extract according to the invention comprises at least 25% of compounds selected from aromadendrene, ledene and delta-cadinene.

In particular, the extract according to the invention may comprise:
from 5% to 15% of aromadendrene,
from 10% to 25% of ledene, and
from 10% to 25% of delta-cadinene.

In another additional embodiment, the extract according to the invention comprises:
less than 6% (for example less than 5%, 4.5%, 4.0%, 3.5%, 3.0%, 2.0%, 1%, 0.5%) of terpinen-4-ol,
less than 5%, (for example less than 2.0% or 1.5%) of alpha-terpineol,
less than 15.0% (for example less than 10.0%, 8.0%, 6.0%, 5.0%, 4.0%, 3.0% or 2.0%) of terpineols,
less than 10% (for example less than 5%, 2%, or 1%) of monoterpenes,
less than 10% (for example less than 5%, de 2%, or 1%) of monoterpenoids other than terpineols,
at least 27% (for example more than 30%, 40%, 50%, 60%, or 70%) of compounds selected from sesquiterpenes and sesquiterpenoids.

Preferably, the extract according to the invention comprises at least 27% of compounds selected from aromadendrene, ledene, delta-cadinene, globulol and viridifloral. In particular, the extract according to the invention may comprise:
from 5% to 15% of aromadendrene,
from 10% to 25% of ledene,
from 10% to 25% of delta-cadinene,
from 1.0% to 5.0% of globulol, and
from 1.0% to 5.0% of viridifloral.

The extract according to the invention may be obtained from an essential oil of Melaleuca alternifolia (referred to hereinafter as “first essential oil” or “starting essential oil”) by fractional distillation.

The starting essential oil is typically rich in terpinen-4-ol and is generally obtained by steam distillation or by hydrodistillation of all or part of Melaleuca alternifolia, preferably of the leaves and/or branches.

In certain embodiments, the starting essential oil of Melaleuca alternifolia may comprise:
from 35% to 48% (for example from 37% to 45%) of terpinen-4-ol,
from 10% to 28% (for example from 14% to 28%) of gamma-terpinene,
from 5.0% to 13% (for example from 6% to 12%) of alpha-terpinene,
from 0.2% to 8.0% (for example from 0.2% to 5.0% or from 1.5% to 8.0%) of alpha-terpineol, and

Preferably, it comprises less than 3.0% of aromadendrene, less than 3.0% of ledene, less than 3.0% of delta-cadinene, less than 1.0% of globulol, and less than 1.0% of viridifloral. The extract of Melaleuca alternifolia according to the invention is advantageously a fraction resulting from the fractional distillation of an essential oil of Melaleuca alternifolia. The fractional distillation is typically carried out under reduced pressure, using a fractional distillation device comprising a boiler, a fractionating column, a condenser and a collector. The essential oil is slowly heated under reduced pressure so as to collect various successive fractions, each fraction corresponding to a temperature step. The fractional distillation is carried out so as to collect firstly at least one (preferably 2, 3 or 4)distillation fractions enriched in monoterpene- and monoterpenoid-type compounds (notably terpin-4-ol). Once the fraction(s) enriched in compounds of monoterpene and monoterpenoid type have been collected, the fraction corresponding to the extract according to the invention can be collected. This fraction is typically obtained under temperature and reduced pressure conditions that make it possible to collect compounds having a boiling point above 200° C. at atmospheric pressure.

Since fractional distillation is a well-known process, a person skilled in the art knows how to determine the heating temperature and the pressure to be used in order to obtain, from a given essential oil, a fraction corresponding to an extract according to the invention, e.g. by carrying out routine tests and/or using nomograms.

In one preferred embodiment, the extract according to the invention is a distillation fraction of an essential oil of Melaleuca alternifolia, preferably obtained by fractional distillation. Thus, the extract according to the invention may also be denoted as being “a distillation fraction of an essential oil of Melaleuca alternifolia” said essential oil preferably being obtained by steam distillation or hydrodistillation of leaves and/or branches of Melaleuca alternifolia. The extract according to the invention is therefore typically an oily extract or an oil of Melaleuca alternifolia. Compared to a conventional essential oil, the extract according to the invention is depleted in monoterpenes and monoterpinoids.

In certain embodiments, said extract of Melaleuca alternifolia is obtained by a process comprising:
i) a step of hydrodistillation of all or part of Melaleuca alternifolia, preferably its leaves and/or ends of branches, so that a first essential oil is obtained,
ii) a step of fractional distillation of said first essential oil under reduced pressure and temperature conditions that make it possible to collect a fraction corresponding to said extract.
   b. Precursor Composition, Cosmetic Ingredient and Cosmetic Composition Comprising the Extract According to the Invention

The extract of Melaleuca alternifolia according to the invention exhibits cosmetic effects and can therefore be used as a cosmetic agent.

In the context the present invention, the expression “cosmetic agent” is understood to mean an agent that exerts a cosmetic effect on the skin.

In the context the present invention, the expression “cosmetic effect” is understood to mean any non-therapeutic effect, which aims to modify and/or improve the appearance of the skin or mucous membranes such as the lips, or to protect them from external attacks (sun, wind, humidity, dryness, chemicals), for example to prevent and/or to correct the phenomena linked to the aging thereof or else to prevent or to treat the effects caused by fatigue on the skin. As will be described in detail below, the extract according to the invention can be used as a regenerative, repairing or anti-fatigue cosmetic agent for the skin. For this purpose, the extract of Melaleuca alternifolia according to the invention is generally introduced as a cosmetic agent into a cosmetic composition. In other words, said extract is typically applied to the skin of the subject to be treated by means of a cosmetic composition. A precursor composition or a cosmetic ingredient comprising the extract of Melaleuca alternifolia according to the invention may be prepared beforehand in order to then be incorporated into the cosmetic composition.

Thus, one subject of the present invention is a precursor composition for the preparation of a cosmetic composition, said precursor composition comprising from 0.1% to 1% by weight, preferably from 0.1% to 0.5% by weight, of the extract of Melaleuca alternifolia according to the invention, in a cosmetically acceptable carrier.

Another subject of the present invention is a cosmetic ingredient comprising from 0.1% to 1% by weight, preferably from 0.1% to 0.5% by weight, of the extract of Melaleuca alternifolia according to the invention, in a cosmetically acceptable carrier.

The cosmetically acceptable carrier may be of any type. For example, it may be a cosmetically acceptable solvent, in particular lower alcohols notably ethanol, isopropanol, dipropylene glycol, butylene glycol, propanediol, glycerol, sorbitol, propylene glycol, pentylene glycol, triheptanoin, an aqueous solution thereof, or a mixture thereof.

Preferably, said carrier is pentylene glycol or triheptanoin.

In a particular embodiment, the cosmetic ingredient or the precursor composition according to the invention consists essentially of, or consists of, an extract of Melaleuca alternifolia according to the invention in a cosmetically acceptable carrier selected from pentylene glycol and tripheptanoin.

The extract of Melaleuca alternifolia according to the invention is preferably present in a content of from 0.1% to 1% by weight, preferably from 0.1% to 0.5% by weight, relative to the total weight of the cosmetic ingredient.

Another subject of the present invention is a cosmetic composition comprising the extract of Melaleuca alternifolia according to the invention, preferably as a cosmetic agent, or the precursor composition or the cosmetic ingredient according to the invention, in combination with at least one cosmetically acceptable excipient.

The cosmetically acceptable excipient(s) present in the composition may be selected from diluents, dispersants, gelling agents, emollients, vectorizing agents (such as polycationic polymers, phospholipids, unilamellar or multilamellar liposomes, niosomes, ethosomes, lamellar systems, nanosomes, lipid or polymer vesicles, nanospheres, microparticles or nanoparticles of natural or non-natural polymers, hydrogels), gums, resins, solvents, in particular lower alcohols, notably ethanol, isopropanol, dipropylene glycol, butylene glycol, propanediol, glycerol, sorbitol, and propylene glycol, fillers such as modified and polymerized starches, titanium dioxide, or a metal stearate, preservatives, essential oils, pearlescent agents, dyes, odor absorbers, pH regulators or neutralizing agents, lubricants, thickeners, hydrotropes such as surfactants, including anionic, cationic, amphoteric or nonionic surfactants, humectants, wetting agents, stabilizers, fillers, dispersants, fragrances, organic or mineral pigments, such as iron oxides, oily agents such as oils or fats of plant origin, fats of animal origin, synthetic oils such as petroleum jelly, silicone oils (cyclomethicone), fatty alcohol esters, fluorinated oils, waxes, modified clays, bentones, metal salts of fatty acids, silica, polyethylenes, mica, preservatives, antimicrobial agents, carriers such as a mineral, spring or floral water, and/or other substances commonly used in formulation in the cosmetics or pharmaceutical field. In one embodiment, the cosmetic composition comprises from 0.0005% to 0.01%, preferably from 0.001% to 0.005% by weight of the extract of Melaleuca alternifolia according to the invention.

More particularly, said cosmetic composition may comprise:
from 0.0005% to 0.01% by weight, preferably from 0.001% to 0.005% by weight of said extract of Melaleuca alternifolia, and
from 99.99% to 99.9995% by weight of one or more cosmetically acceptable excipients.

In another embodiment, said cosmetic composition comprises from 0.5% to 5% by weight, preferably from 1% to 3% by weight, of said precursor composition or of said cosmetic ingredient.

More particularly, said cosmetic composition may comprise:
0.5% to 5% by weight, preferably from 1% to 3% by weight of said precursor composition or of said cosmetic ingredient, and
from 95% to 99.5% by weight of one or more cosmetically acceptable excipients.

In certain embodiments, the cosmetic composition further comprises one or more additional active agents having a cosmetic effect. The expressions “active ingredient having a cosmetic effect” or “active agent having a cosmetic effect” or “cosmetic or active agent having a cosmetic effect” is understood to mean a compound capable of exerting at least one cosmetic effect on the skin or appendages thereof. The expression “cosmetic effect” is understood to mean any nontherapeutic effect, which aims to modify and/or improve the visual appearance and mechanical properties of the skin or mucous membranes such as the lips, to protect them from external attacks (sun, wind, humidity, dryness, chemicals), to prevent and/or to correct the phenomena linked to the aging thereof or else to prevent or to treat the effects caused by stress or fatigue on the skin.

Examples of such agents are, inter alia, antiwrinkle agents, anti-aging agents, antioxidants, moisturizing agents, soothing agents, anti-redness agents, decongestants, scrubbing or exfoliating agents, matting agents, sebum regulators, lightening active agents, anti-dark spot active agents, agents for combating dark circles and bags, antistress agents, antifatigue agents, anti-pollution active agents, tautening agents, sunscreens and sunblocks, and combinations thereof.

Notably, the cosmetic composition may comprise tocopherols, and/or plant extracts such as linseed extracts, Vibrio exopolysaccharide extracts, peptides such as trifluoroacetyl tripeptide-2.

In one particular embodiment, the cosmetic composition may comprise an active agent selected from an antiwrinkle agent, an anti-redness agent, an antioxidant, a moisturizing agent, a soothing agent, a sebum regulator, an anti-dark spot or lightening agent, a tautening agent, an anti-pollution active agent, a dark circle or puffiness corrector, a sunscreen or sunblock, and combinations thereof.

As examples of anti-pollution agents, mention may be made of extracts of Chrysanthellum indicum, polysaccharides, notably marine polysaccharides from a Alteromonas fermentation medium and Nigari salts.

As examples of dark circle correctors, mention may be made of Chrysantellum Indicum extracts or else the extracts and sulfated polysaccharides of algae, notably of Ascophyllum nodosum or of Asparagopsis armata.

As examples of moisturizing agents, mention may be made of urea, pidolic acid (PCA) and derivatives thereof, in particular salts thereof such as arginine PCA, chitosan PCA, copper salts (copper PCA), magnesium salts (magnesium PCA), sodium salts (sodium PCA) or zinc salts thereof, ethylhexyl PCA, calcium gluconate, hyaluronic acid and salts thereof and other glycosaminoglycans, fructose, glucose, isomaltose, lactose, trehalose, polydextrose, saccharose (sucrose), maltitol, mannitol, sorbitol, xylitol and other carbohydrates and derivatives, polyethylene glycols such as PEG-7, PEG-8, PEG-10, PEG-12 or PEG-14, glycerol, propylene glycol, butylene glycol, betaine, citrulline, collagen and derivatives thereof, histidine, silk hydrolysates, keratin hydrolysates or soy hydrolysates, plant extracts rich in polysaccharides and/or polyphenols, for example extracts of aloe, cornflower ((′entaurea cyanus), extracts rich in polysaccharides, notably resulting from fermentation media of marine microorganisms such as Alteromonas and the combinations thereof.

As examples of anti-ageing agents, mention may be made of ascorbic acid and derivatives thereof, such as magnesium ascorbyl phosphate, glycosaminoglycans and derivatives thereof, (′yathea polysaccharides, collagen, linseed (Linum usitatissimum) extracts, peptides such as caprooyl tetrapeptide-3 and trifluoroacetyl tripeptide-2, extracts of Polygonum aviculare, extracts of brown algae, in particular of Ascophyllum nodosum, extracts of ferns, in particular of (′yathea cumingii.

As examples of antistress agents, mention may be made of the Rosality™ products (combination of a rose water and of a rose essential oil) and the extract of pink-flowered cistus, for example sold under the brand IBR-Chill™

As examples of soothing agents, mention may be made of allantoin, extracts of aloes, of birch (for example Betula alba), of willowherb (Epilobium angustifolium), of chestnut (for example (′astenea sativa), of cornflower (for example (′entaurea cyanus), of centella (for example (′entella asiatica), of horsetail (for example Equisetum arvense), of fennel (for example Foeniculum vulgare), of witch hazel (for example Hamamelis virginiana), of ivy (for example Hedera helix), of Habiscus sabdariffa, of lily (for example Lilium candidum), of mallow (for example Malva sylvestris), of lemon balm (for example Melissa officinalis), of skullcap (for example Scutellaria baicalensis), of mimosa (for example Mimosa temuflora), of cinquefoil (for example Potentilla erecta), an extract of oligosaccharides or an oligosaccharide, for example of flax, peptides such as palmitoyl tripeptide-8, extracts of polysaccharides, notably the extracts of exopolysaccharides resulting from the Alteromonas fermentation medium and combinations thereof.

As examples of antioxidants, mention may be made of HMR (hydroxymethyl resorcinol), ascorbic acid and derivatives thereof, vitamin B9, histidine hydrochloride, or an extract of willowherb (Epilobium augustifolium). The active ingredients having an antioxidant effect and of vitamin type are generally used at a weight percentage of at least 1% relative to the total weight of the cosmetic composition.

As examples of sebum regulators, mention may be made of flaxseed lignans, rice powder, zinc gluconate, sarcosine, an extract of (′innamomum zeylanicum bark, an extract of avocado, an extract of Backhousia citriodora and combinations thereof.

As anti-redness agents, mention may be made of saponins, flavonoids, ruscogenins, esculosides, and extracts containing them, for example extracts of Ruscus, and also certain essential oils, for example of lavender or of rosemary.

As examples of anti-dark spot agents, mention may be made of extracts such as licorice (Glycyrrhyza glabra), extract of jackfruit (Artocarpus heterophyllus), extracts of dock (R. occidentalis), extracts of a plant belonging to the Citrus genus, resveratrol, peptides such as oligopeptide-68, nonapeptide-1, kojic acid, magnesium ascorbyl phosphate and combinations thereof.

In one particular embodiment, said cosmetic composition comprises from 0% to 30% by weight, for example from 0 to 15% by weight, of one or more additional cosmetic active agents. In one more particular embodiment, said cosmetic composition comprises:
from 0.0005% to 0.01% by weight, preferably from 0.001% to 0.005% by weight of said extract of Melaleuca alternifolia according to the invention,
from 69.99% to 99.9995% by weight of one or more cosmetically acceptable excipients, and
from 0% to 30% by weight, for example from 0 to 15% by weight, of one or more additional cosmetic active agents.

In one more particular embodiment, said cosmetic composition comprises:
0.5% to 5% by weight, preferably from 1% to 3% by weight of said precursor composition or of said cosmetic ingredient as described above,
from 65% to 99.5% by weight of one or more cosmetically acceptable excipients, and
from 0% to 30% by weight, for example from 0 to 15% by weight, of one or more additional cosmetic active agents.

The extract of Melaleuca alternifolia according to the invention, the precursor composition or the cosmetic ingredient comprising, may be incorporated into any type of cosmetic composition. Preferably, it is a cosmetic composition having a form suitable for topical administration, in particular suitable for application on the skin. The cosmetic composition according to the invention may be of any type. In one embodiment, the cosmetic composition is selected from the group consisting of aqueous solutions, aqueous-alcoholic solutions, oil-in-water (O/W) emulsions or water-in-oil (W/O) emulsions or multiple (triple: W/O/W or O/W/O) emulsions, nanoemulsions, in particular O/W nanoemulsions, the drop size of which is typically less than 100 nm, aqueous gels, or spherule-assisted dispersions of a fatty phase in an aqueous phase, suspensions, preferably in aqueous or aqueous-alcoholic media, liposome suspensions, powders, lotions, milks, creams, unguents, gels, foams, balms and ointments.

In some embodiments, the cosmetic composition according to the invention is a cream, a balm, a mask, a serum or a lotion.

In one preferred embodiment, the cosmetic composition according to the invention is a serum, cream, a mask or a balm, preferably for use at night.

c. Cosmetic Uses and Methods According to the Invention

According to an additional aspect, another subject of the invention is the use of the extract of Melaleuca alternifolia as described above as a regenerative, repairing or anti-fatigue cosmetic agent for the skin, and more particularly for treating or preventing one or more signs of skin fatigue.

Another subject of the invention is the use of a cosmetic composition according to the invention for treating or preventing one or more signs of skin fatigue.

For the purpose of the invention, the terms “skin” and “cutaneous” refer to any part of the skin of the human body, in particular the skin of the face, including the lips and the eyelids, the neck, and the skin of the hands. Preferably, it is the skin on the face, including the area around the eyes and the area around the lips, and on the neck.

In the context of the present invention, the extract or the composition according to the invention is typically applied topically, preferably on skin or a mucous membrane. This is healthy skin or a healthy mucous membrane, i.e. skin or a mucous membrane that does not have any wounds or cutaneous pathologies. In particular, the extract or the composition according to the invention is applied to non-acne skin (i.e. skin not suffering from acne) that does not have any wounds, notably resulting from insect bites, or any skin infections, e.g. caused by a fungus (mycosis), a bacterium or a protozoa.

In other words, the extract or the composition according to the invention does not produce a therapeutic effect on the skin when it is used according to the invention.

The expression “anti-fatigue agent” is understood to mean a cosmetic agent capable of preventing, of treating or of reducing the “cutaneous effects or signs” of fatigue, i.e. the effects of fatigue on the skin.

In the context of the present invention, the expression “cutaneous effects or signs” is understood to mean any non-pathological alteration or modification of the visual appearance or mechanical properties of the skin. In the context of the present invention, the “cutaneous effects or signs” are caused by, or associated with, fatigue.

The expression “preventing a sign or an effect” is understood to mean the fact of slowing down or preventing the appearance of said sign or effect on the skin. The expression “treating a sign or an effect” is understood to mean the fact of decreasing, reducing, toning down, correcting or slowing down the development of said sign or effect on the skin.

The sign(s) of skin fatigue may for example be a loss of radiance of the skin, a dull complexion, a peaky complexion, a loss of uniformity of the complexion, haggard facial features, dark circles around the eyes, bags under the eyes, puffy eyelids, an alteration in the smooth appearance of the skin, an increase in the roughness of the skin, a loss of elasticity, a loss of density, a loss of firmness, a loss of hydration, appearance of fine lines or wrinkles, appearance of redness, and combinations thereof.

In the context the present invention, the fatigue that causes non-pathological modifications or alterations of the skin may be of any type. It may be temporary or chronic fatigue. It may notably be fatigue caused by one or more factors, notably environmental factors. These may be factors such as lack of sleep, for example due to the work rhythm or the family situation (e.g. birth of a child), sleep disorders or jet-lag, poor or unbalanced dietary health, alcohol consumption, lack of or excess physical activity, sedentary lifestyle, overwork, intense activity or inadequate activity at work, night work or staggered working hours, significant screen exposure, stress, anxiety or worries linked to professional or family life.

Fatigue may also result from physiological situations such as pregnancy or menopause. Preferably, fatigue is not associated with or caused by a pathological state in a subject. In certain embodiments, the extract of Melaleuca alternifolia according to the invention may be used, for at least one (1, 2, 3, 4, 5, 6, 7, 8, 9 or 10) of the following purposes:
preventing, treating or reducing dark circles and/or bags under the eyes,
preventing, treating or reducing puffiness of the eyelids,
preventing, treating or reducing pigmentation irregularities of the skin, including the pigment spots induced by fatigue,
homogenizing or unifying the complexion of the face,
making the complexion of the face more luminous and/or more radiant and/or clearer,
preventing or treating a dull complexion, a peaky complexion and/or haggard features,
making the skin, notably on the face, fresher and more luminous,
soothing the facial features,
making the appearance of the skin less tired, more relaxed, fresher,
making the gaze less tired, more rested and more luminous,
promoting or accelerating the recovery of the epidermis, notably of the face,
revitalizing the skin, notably of the face,
promoting a bright complexion effect,
preventing, treating the alteration of, or restoring the barrier function of the skin,
preventing, treating or reducing a loss of firmness of the skin, notably of the face,
preventing, treating or reducing the dehydration of the skin (or in an equivalent manner “loss of hydration”),
preventing, treating, slowing down, or reducing the appearance of wrinkles or fine lines, and/or
preventing, treating or reducing the appearance of redness.

It goes without saying that the non-pathological changes listed above are associated with or caused by fatigue on the skin.

In certain embodiments, the extract according to the invention can be used as a cosmetic agent to prevent, treat or reduce one or more signs of skin fatigue selected from a dehydration of the skin, a loss of elasticity, a loss of firmness, wrinkles, and combinations thereof.

In the context of the present invention, the expression “regenerative agent or repairing agent” is understood to mean a cosmetic agent capable of promoting or stimulating the regeneration of the skin or the repair of the skin during sleep so that the cutaneous effects caused by fatigue are reduced, treated or prevented. It may notably be chronic fatigue or fatigue caused by one or more factors, notably environmental factors. In this regard, the applicant has notably observed that the extract according to the invention stimulates the genes involved in the synthesis of collagen in the skin.

As is illustrated in the examples, the application of the extract on the skin at a very low concentration, notably that cannot be detected by the sense of smell, makes it possible to improve sleep.

It has also been demonstrated that the extract according to the invention is capable of activating the signaling pathways of melatonin, notably those involved in the mechanisms for DNA repair, and cell defence against oxidative stress but also in the synthesis and transportation of melatonin, for example induced by exposure to an environmental stress such as excessive exposure to UV rays.

Thus, in one particular embodiment, the extract according to the invention is used as an agent for activating the signaling pathway of melatonin and/or potentiating the effect of melatonin on the skin.

According to one particular aspect, the extract according to the invention is used to promote or improve skin recovery, notably during sleep. The extract according to the invention is notably used to potentiate the natural mechanisms for repairing and regenerating the skin which are controlled by melatonin.

In one additional or alternative aspect, the extract according to the invention is used to improve sleep, notably to increase the total duration of sleep and/or that of deep sleep.

According to one additional embodiment, the extract according to the invention is used to promote or favor skin recovery during the sleep phase.

According to one additional embodiment, the extract according to the invention is used to repair/regenerate the skin of a subject exposed to fatigue, notably to chronic fatigue.

The extract according to the invention may be used to repair/regenerate skin exposed to a stress, notably an environmental stress such as exposure to sunlight, to wind, to humidity, to dryness or to chemicals or processes of abrasion, rays, etc. type or else to stress linked to lifestyle (jet lag, lack of sleep, night work, etc.).

Additionally or alternatively, the extract according to the invention is used to induce the synthesis of collagen in the skin.

Additionally or alternatively, the extract according to the invention can also be used as an agent for reducing the formation of reactive oxygen species (ROS) and/or to stimulate the expression of antioxidant proteins, in particular antioxidant enzymes in the skin.

In one particular embodiment, the extract of Melaleuca alternifolia according to the invention is present as a cosmetic active agent in a cosmetic composition, preferably a cream, a balm, a mask, a serum or a lotion, as is described above. The concentration of the extract Melaleuca alternifolia according to the invention in such a cosmetic composition is advantageously low, so that it cannot be detected by the sense of smell. Typically, this concentration is from 0.0005% to 0.01% by weight, preferably from 0.001% to 0.005% by weight of said extract.

It goes without saying that such uses are intended for individuals exposed to fatigue as defined above.

The individuals targeted by the present invention may be of any age and gender. In a preferred embodiment, it is a subject of less than 65 years old. It may be for example a woman or a man of from 25 to 65 years old, notably from 25 to 55 years old. By way of example, it may be a woman aged from 25 to 45 years old.

An additional subject of the invention is a cosmetic method for preventing or treating the signs of skin fatigue in a subject, said method comprising the application of a cosmetically effective amount of an extract of Melaleuca alternifolia according to the invention, or of a cosmetic composition according to the invention on the skin.

The objective of the cosmetic method according to the invention is to treat or prevent one or more cutaneous effects of fatigue, preferably selected from an alteration in the complexion of the skin, notably a peaky complexion, a dull complexion, a loss of radiance of the complexion or a loss of uniformity of the complexion, notably the appearance of pigment spots, haggard facial features, the appearance of bags and/or dark circles around the eyes, puffy eyelids, an alteration in the smooth appearance of the skin, an increase in the roughness of the skin, a loss of elasticity, a loss of density, a loss of firmness, a loss of hydration, an appearance of wrinkles, an appearance of redness, and combinations thereof.

The method according to the invention may make it possible to obtain any one of the following effects, in a subject exposed to fatigue:
preventing, treating or reducing dark circles and/or bags under the eyes,
preventing, treating or reducing puffiness of the eyelids,
preventing, treating or reducing pigmentation irregularities of the skin, including the pigment spots induced by fatigue,
homogenizing or unifying the complexion of the face,
making the complexion of the face more luminous and/or more radiant and/or clearer,
preventing or treating a dull complexion, a peaky complexion and/or haggard features,
making the skin, notably on the face, fresher and more luminous,
soothing the facial features,
making the appearance of the skin less tired, more relaxed, fresher,
making the gaze less tired, more rested and more luminous,
promoting or accelerating the recovery of the epidermis, notably of the face,
revitalizing the skin, notably of the face,
promoting a bright complexion effect,
preventing, treating the deterioration of, or restoring the barrier function of the skin,
preventing, treating or reducing a loss of firmness of the skin, notably of the face,
preventing, treating or reducing the dehydration of the skin (or in an equivalent manner “loss of hydration”),
preventing, treating, slowing down, or reducing the appearance of wrinkles, and/or
preventing, treating or reducing the appearance of redness.

In the cosmetic methods and uses according to the invention, the dose to be administered and the frequency of administration of the combination according to the invention vary as a function of the desired cosmetic effect, characteristics of the individual, in particular the sex, age and skin type thereof. Typically, the extract of Melaleuca alternifolia according to the invention, or the cosmetic composition comprising same, may be applied to the area to be treated once a day, preferably before going to bed, for several consecutive weeks or even several months, for example at least for 3 months. By way of example, the patient may apply a dose of 1 g to 2 g of cosmetic composition to their face in the evening.

### General Definitions:

Unless otherwise indicated, all the technical and scientific terms used here have the same meaning as that commonly understood by those skilled in the art in the technical field of the present application.

This application is not limited by the examples of methods, uses and compositions described herein, and all methods, uses and compositions similar or equivalent to those described herein can be used in the embodiments of the present application.

The section headers and titles provided herein should not be considered to be limitations of the various aspects or embodiments of the present application. The section headers and titles are used here for organizational purposes only and should not be construed as limiting the invention described. All the terms defined are more fully defined by reference to the application as a whole.

All publications, including patent documents and scientific articles, mentioned in this application are incorporated by reference in their entirety to the same extent as if each individual publication were incorporated individually by reference. The citation of these publications should in no way be construed as an admission that such publications constitute prior art. If a definition stated in the present application is contrary to or inconsistent with a definition stated in the patents, applications, published applications and other publications incorporated herein by reference, the definition stated in the present application prevails over the definition incorporated herein by reference.

All the features described in this application can be combined in any combination. Each feature described in the present application may be replaced by another feature having the same objective, or an equivalent or similar objective. Thus, unless expressly stated otherwise, each feature disclosed is only one example of a generic series of equivalent or similar features.

Definitions of terms may appear throughout the description. It should be understood that this description is not limited to the particular embodiments described, since these may, of course, vary. It should also be understood that the terminology used herein has the sole purpose of describing particular embodiments and is not intended to be limiting.

It should be noted that, as used here and in the appended claims, the singular forms “a (n)”, “one” and “the” include plural referents unless the context clearly indicates otherwise. For example, “a (n)” or “one” includes “at least one” and “one or more”.

The terms “comprising”, “comprises” and “composed of” as used herein are synonymous with “including”, “includes”, “containing”, “contains”, and grammatical variants thereof. These terms are inclusive or open and do not exclude the presence of additional members, elements or process steps that are not mentioned.

When a range of values is provided, it is understood that each intermediate value between the upper and lower limits of this range is also specifically disclosed and encompassed in this description.

### EXAMPLES

The invention will be better understood in light of the following examples, which are given purely by way of illustration and do not have the aim of limiting the scope of the invention.

### Example 1: Preparation of an Extract According to the Invention

### 1. Production of an Extract of Melaleuca Alternifolia According to the Invention

As indicated in the description, the extract according to the invention may be obtained by fractional distillation of a standard essential oil of Melaleuca alternifolia. The essential oil was obtained from fresh leaves and terminal branches by steam distillation. The essential oil was then fractionated by distillation. A first fraction rich in gamma-terpinene was obtained, then a second fraction rich in both gamma-terpinene and terpinen-4-ol, followed by a third fraction and a fourth fraction, both rich in terpinen-4-ol. Lastly, the extract according to the invention, corresponding to the fifth fraction, was obtained.

Table 1 gives details of the physical and chemical characteristics of the essential oil of Melaleuca alternifolia steam-distilled before the fractional distillation. Table 2 gives details of the composition of the starting material (“first essential oil”) and those of the fraction of interest (“essential oil according to the invention”).

GC/FID analyses were carried out on a Agilent 6890 machine (Agilent Technologies) equipped with a flame ionization detector (FID), with an automatic liquid sampler and with the same HP-1 MS capillary column (100% dimethylpolysiloxane, 60 m×0.15 mm i.d., film thickness 0.25 μm; Agilent Technologies). The temperature of the oven was maintained at 60° C. for 10 min, then programmed from 60° C. to 300° C. with 2° C./min, and finally 300° C. for 10 m (passage time 140 min). The temperatures of the injector and of the detector were respectively 250° C. and 300° C.; H₂ (1.0 ml/min) as carrier gas; injection in split mode (1:50); volume injected 0.1 ml. The flow rates of O₂, H₂ and makeup gas of the FID were 350, 35 and 20 ml/min, respectively.

TABLE 1
Characteristics of the starting essential oil
PHYSICOCHEMICAL CHARACTERISTICS OF THE STARTING ESSENTIAL OIL

| | |
|---|---|
| APPEARANCE | LIQUID |
| COLOR | COLORLESS-LIGHT YELLOW |
| ODOR | CHARACTERISTIC |
| RELATIVE DENSITY (20° C./20° C.) | 0.885-0.906 |
| REFRACTIVE INDEX (20° C.) | 1.475-1.482 |
| ROTATORY POWER (20° C.) | +5° TO +15° |
| MOISTURE (VISUAL) | NO WATER VISIBLE AT 20° C. |
| MISCIBILITY IN 85% (V/V) ETHANOL | 1 VOLUME OF OIL GIVES A CLEAR SOLUTION IN |
| AT 20° C. | AT MOST 2 VOLUMES OF 85% (V/V) ETHANOL |
| FLASH POINT | 50° C.-60° C. |

TABLE 2
Composition of volatile compounds of the starting essential
oil and of the extract according to the invention.

| | CONTENT % (GC-FID AREA) | |
|---|---|---|
| | STARTING | EXTRACT ACCORDING |
| COMPOUNDS | ESSENTIAL OIL | TO THE INVENTION |
| alpha-Pinene | 1.0-6.0 | — |
| Sabinene | UP TO 3.5 | — |
| alpha-terpinene | 5.0-13.0 | 0.02 |
| Limonene | 0.5-1.5 | — |
| para-Cymene | 0.5-4.0 | 0.22 |
| 1,8-Cineole | UP TO 5.0 | — |
| gamma-Terpinene | 10.0-28.0 | 0.01 |
| alpha-Terpinolene | 1.5-5.0 | 0.01 |
| Terpinen-4-ol | 37.0-45.0 | 0.16 |
| alpha-Terpineol | 1.5-8.0 | 1.09 |
| Aromadendrene | UP TO 3.0 | 8.65 |
| Ledene | UP TO 3.0 | 17.80 |
| delta-Cadinene | UP TO 3.0 | 17.29 |
| Globulol | UP TO 1.0 | 3.25 |
| Viridifloral | UP TO 1.0 | 2.61 |

### Example 2: Evaluation of the extract according to the invention on skin explants

Protocol: The objective of this study was to evaluate the repairing effect of the extract according to the invention on skin explants and to demonstrate its “melatonin-like” activity on the skin. The extract according to the invention obtained in example 1 was diluted in pentylene glycol to a concentration of 0.5% by weight. This precursor composition (also referred to as cosmetic ingredient) was then formulated in carbopol at a concentration of 1% by weight. It is this final composition which was used in this experiment.

On an abdominoplasty originating from a Caucasian woman of 46 years old (reference: P2471-AB46, of phototype II-III (according to the Fitzpatrick skin color classification), 51 explants having a mean diameter of 11 mm (+1 mm) were prepared. The explants were kept alive in a BEM medium (BIO-EC's Explants Medium) at 37° C. in a moist atmosphere containing 5% of CO₂ for 2 days. On the 2^{nd} day, the culture medium was replaced with an HBSS solution then the explants were irradiated with UV rays (“UV-Placebo” group and “UV-Extract” group) with a dose of 13.5 J/cm² of UVA corresponding to 3 MED (minimal erythema dose) and with a dose of 0.15 J/cm² of UVB corresponding to 1 MED or without this irradiation (“Placebo” group). At the end of the irradiation, the placebo product (for the “Placebo” group and “UV-Placebo”group) or the composition comprising the extract according to the invention (“UV-extract”) were applied topically to the cutaneous surface of the corresponding explants in a proportion of 2 μl per cm² explant (˜ 2 mg/cm²) for an additional 24 h. On Day 3, the explants were fixed in RNAlater before extraction of the RNA using ReliaPrep™ RNA Tissue Miniprep System (fibrous) from Promega. The quality and the concentration of the RNAs were evaluated before carrying out the reverse transcription step with iScript (Bio-Rad, 20 μl/reaction). With the aid of a spike control, the ΔCq was calculated. The benchmarks evaluated for each qPCR reaction using the CFX Manager 3.1 software are the following:
The logarithmic curve of the amplification for each sequence of interest.
The melting curves are fluorescence intensities recorded after the last cycle. The temperature goes from 65° C. to 96° C. with an increment of 0.5° C. every 5 seconds.
The curves representing the inverse of the derivative of the fluorescence signal as a function of temperature (in° C.) make it possible to verify a single amplicon.

All the samples were subjected to an analysis of the gene expression profile. For the quantification, the number of cycles was normalized relative to the reference gene B2M:

$$Cq ( relative amount ) per sample and per gene = E ( gene ) ( Cq ( control ) - Cq ( treated ) ) \qquad ( i )$$

$$E = efficiency of the pairs of primers = ( \% efficiency * 0.01 ) + 1$$

$$Cq ( control ) = the quantitative threshold calculated by the \\ \\ \\ CFX Maestro in regression mode for the control condition .$$

$$Cq ( treated ) = the quantitative threshold calculated by the \\ \\ \\ CFX Maestro in regression mode for the treated condition .$$

$$gene = target gene$$

$$Expression of a normalized target gene = NE \qquad ( ii )$$

$$NE = Expression of normalized sample ( gene ) = Cq sample ( gene ) / ( {( Cq sample ( ref 1 ) * Cq sample ( ref 2 ) )}^{1 / n}$$

$$Cq = relative amount the denominator corresponds to \\ \\ \\ the normalization factor including the two reference genes : ref$$

$$1 = B 2 M$$

$$gene = target gene$$

For each gene of interest, the values were calculated between the treatment samples in triplicate relative to the control samples of matched explants in triplicate for each kinetic time point. A gene was considered to be induced if its expression showed an increase of greater than or equal to 1.5 relative to the control (fold-change≥1.5). Similarly, a gene was considered to be repressed if it exhibited a reduction in its expression relative to the control (fold-change≤0.65).

Furthermore, when a homogeneous gene modulation is maintained between the biological triplicates, a P value<0.05 (according to the T test) is indicated by an asterisk.

Results: Compared to the UV with placebo (UV-placebo) condition, the extract triggered a strong induction of OGGI which is involved in DNA repair. Furthermore, the extract induced a strong induction of antioxidant enzymes such as CAT and GPX1. An effect on COL3A1 and COL1A2 also tends to prove that the extract according to the invention acts on the extracellular matrix with an increase in the synthesis of collagen. Interestingly, the extract induced an increase in the expression of genes associated with melatonin (ASMTL, RORα, SIRT3 and SLC15A1) which tends to prove that the extract targets the same pathway as melatonin and is therefore capable of activating the signaling pathways of melatonin, notably those involved in the mechanisms for protection against oxidative stress, notably induced by UV rays. The stimulation of this signaling pathway would explain the stimulation or repression of the genes mentioned above.

All of these results support the use of the extract according to the invention as an antifatigue, regenerative or repairing agent for the skin.

TABLE 3
Gene expression results

| | UV-Extract vs | |
|---|---|---|
| | UV-placebo | P-value |
| ASMTL | 1.55 | 0.042 |
| RORα | 1.63 | 0.040 |
| SIRT3 | 1.62 | 0.008 |
| SLC15A1 | 2.62 | 0.007 |
| CAT | 1.94 | 0.041 |
| GPX1 | 1.70 | 0.033 |
| COL3A1 | 2.56 | 0.013 |
| COL1A2 | 1.97 | 0.012 |
| OGG1 | 2.02 | 0.005 |

### Example 3: Improvement in Sleep

Protocol: 32 healthy participants (16 men, 22-56 years old, median age 41.5 years old) with self-declared minor sleep disorders, caused by factors linked to lifestyle, but without a clinical sleep disorder diagnosis, were selected.

The study consisted of the evaluation of 2 samples of face cream, 1 “active” cream and 1 “placebo” cream, used separately over 3 weeks:
Product A-“active” cream: fragrance-free generic face cream comprising 1% of a precursor composition comprising 0.25% by weight of the extract from example 1, in pentylene glycol (A-Leen® 5, Minasolve),
“Placebo” cream: fragrance-free generic (cf. example 5, table 5 below) face cream comprising 1% of pentylene glycol (A-Leen* 5, Minasolve) only.

Devices: for objective sleep measurements, the participants used the SleepScore Max device (SleepScore Labs, Carlsbad, CA), a contactless monitoring device which uses the respiratory signal and the detection of movement to detect sleep and which was validated by polysomnography (PSG), a standard method for measuring sleep. All the self-declaration data was recorded using Compusense, an online data collection software.

### Study Design and Procedure:

Contextual, randomized and counterbalance study using a fragrance-free control cream.
The participants applied the creams at bedtime, in their natural sleep environment.
The study lasted from Sunday evening to Friday morning for 3 consecutive weeks.
The participants used a different sample each week. The control sample was used during the second week. Table 3 shows an overview of the design of the study.
Each night of the study the participants filled out daily questionnaires and monitored their sleep using the SleepScore Max device.

TABLE 4
Clinical trial details

| | Participants | Sample tested |
|---|---|---|
| Week 1 | Group (N = 16) | Extract according to the invention |
| Week 2 | Group (N = 32) | Placebo |
| Week 3 | Group (N = 16) | Extract according to the invention |

### Data Analytics:

The objective and self-declared data on nocturnal sleep were analysed using the JMP Pro (version 15) statistical software using a mixed model, among the participants. An alpha level of 0.05 was used for all the statistical tests. The comparison was made between the control and the extract. The percentages originate from calculation relative to the control condition.

### Statistical Methods:

The data obtained and the variations as a percentage were subjected to the two-way Student's t-test for paired data. The statistical significance value is p<0.05.

### Results:

The extract according to the invention significantly increased the total duration of sleep (p<0.05) compared to the control, in particular the duration of deep sleep (p<0.05). On average the participants slept for 6.5 hours (392 minutes) per night with the fragrance-free control cream versus 6.7 hours (402 minutes) with the cream containing the extract according to the invention. The duration of deep sleep also went from 1.2 hours (73 minutes) with the control cream to 1.3 hours (79 minutes) with the cream containing the extract according to the invention (p<0.05).

### Example 5: Cosmetic Products Incorporating the Extract According to the Invention

A) Night cream comprising 1% of a precursor composition (also referred to as cosmetic ingredient) comprising 0.25% by weight of the extract according to the invention prepared according to example 1 in pentylene glycol

TABLE 5
Composition of a night cream

| | | % by |
|---|---|---|
| Trade name | INCI | weight |
| DEIONIZED WATER | AQUA (water) | 71.65 |
| CARBOPOL ULTREZ 20 | ACRYLATES/C10-30 | 0.30 |
| | ALKYL ACRYLATE | |
| | CROSSPOLYMER | |
| ARLACEL 165 | GLYCERYL STEARATE | 6.00 |
| | (and) PEG-100 | |
| | STEARATE | |
| DUB ININ | ISONONYL ISONONANOATE | 15.00 |
| LIPEX SHEA ™ | Shea butter | 5.00 |
| | (BUTYROSPERMUM PARKII | |
| | (SHEA) BUTTER) | |
| DEKABEN C4 | PHENOXYETHANOL (and) | 1.00 |
| | METHYLPARABENE (and) | |
| | ETHYLPARABENE (and) | |
| | BUTYLPARABENE (and) | |
| | PROPYLPARABENE | |
| SODIUM HYDROXIDE | SODIUM HYDROXIDE | 0.05 |
| “Extract” in A-Leen-5 ® | Pentylene glycol | 1.00 |
| | and tea tree oil | |
| | | 100.00 |

B) Antifatigue cream comprising 1% of a precursor composition comprising 0.25% by weight of the extract according to the invention prepared according to example 1 in triheptanoin.

TABLE 6

| | | % by |
|---|---|---|
| Trade name | INCI | weight |
| DEIONIZED WATER | AQUA (water) | 79.30 |
| SATIAXANE ™ VPC 911 | XANTHAN GUM | 0.50 |
| DERMOFEEL ® GSC | GLYCERYL STEARATE | 2.00 |
| | CITRATE | |
| DERMOFEEL ® PS | POLYGLYCERYL-3 | 2.00 |
| | STEARATE | |
| SWEET ALMOND OIL | PRUNUS AMYGDALUS | 3.00 |
| | DULCIS OIL | |
| LANETTE ® 16 | CETYL ALCOOL | 2.00 |
| LIPEX SHEASOFT ™ | BUTYROSPERMUM PARKII | 3.00 |
| | BUTTER | |
| CETIOL ® C5 | COCO-CAPRYLATE | 3.00 |
| VITAPHEROLE ® E-1000 | TOCOPHEROL (and) | 0.20 |
| | HELIANTHUS ANNUUS | |
| | (sunflower) SEED OIL | |
| DERMOSOFT ® 1388 | GLYCERIN (and) SODIUM | 4.00 |
| | ANISATE (and) SODIUM | |
| | LEVULINATE (and) AQUA | |
| | (water) | |
| “Extract” in triheptanoin | Triheptanoin and | 1.00 |
| | tea tree oil | |
| | | 100.00 |

### Table 6: Composition of an Antifatigue Cream

### Example 6: Improvement in the Signs of Skin Fatigue

Protocol: A clinical trial was carried out to confirm the antifatigue effect of the extract according to the invention. Forty-four volunteers were divided evenly into two groups (22 individuals in the test group: “Active” cream and 22 individuals in the placebo group: Placebo cream).

The volunteers enrolled were people working staggered hours, young adults who were very socially active and used to going out and going to bed late or traveling (jet lag), or young mothers. The volunteers therefore had disturbed sleep cycles but were not being treated for sleep disorders.

The study was performed for 28 days with measurements on DO, T12 h, D7 and D28. The products were applied once a day by the volunteers, in the evening before going to bed.

The 2 creams tested are:
“Active” cream: fragrance-free generic face cream comprising 1% of a cosmetic ingredient comprising 0.25% by weight of the extract from example 1, in triheptanoin (cf. example 5, table 5 above),
“Placebo” cream: fragrance-free generic (cf. example 5, table 5 below) face cream comprising 1% of triheptanoin only.

Devices: to observe the signs of fatigue, the following parameters were measured:
the transepidermal water loss (TEWL), evaluated by means of the Tewameter® TM 300 (Courage+Khazaka, Electronic GmbH) measurement.
skin hydration, evaluated with a Corneometer*
the antioxidant potential of the skin, measured from samples of the first layers of the stratum corneum taken on D0 and D28 from the cheeks by the experimenter using Corneofix® (Courage+Khazaka, Electronic GmbH). The FRAP (Ferric Reducing Antioxidant Parameter) test is then carried out.
crow's feet wrinkles, measured with Primos 3D (GFMesstechnik GmbH).
Skin elasticity and firmness, measured by the suction/elongation method (Cutometer®MPA 580, Courage+Khazaka, Electronic GmbH). This device applies a negative pressure (suction) where the skin is sucked into the measuring cup of the probe which allows its penetration depth to be measured by an optical measuring system.
The evaluation of the dark circles, from original images taken with the Bio blue light scanner for each volunteer and processed by specific software, was measured using the individual typology angle (ITA°).

### Statistical Method:

For each time, a statistical study was carried out to analyze the significance of the evolution of the parameters. The data obtained and the percentage variations were subjected to a two-way A-NOVA. The statistical significance value is p<0.05 (*p<0.05; ** p<0.01; *** p<0.001; **** p<0.0001).

### Results:

The results of this clinical trial are illustrated in FIGS. 4 to 10.

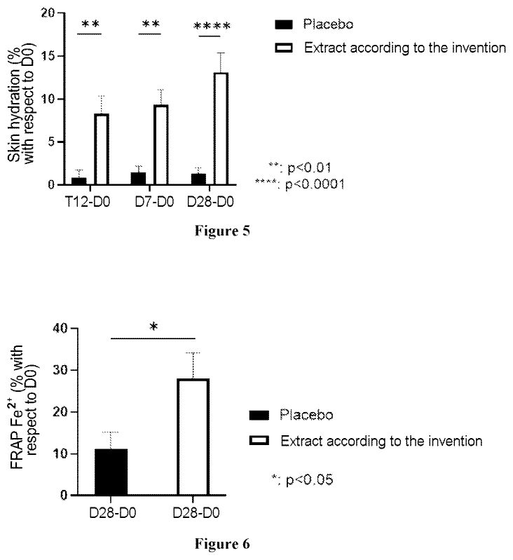

The group treated with the “active” cream exhibits a significant decrease in the transepidermal water loss (TEWL) of the skin, which increases over time, compared with the group treated with the “placebo” cream. This highlights an improvement in the barrier function (FIG. 4). Significant improvement in skin hydration was also observed in the group treated with the “active” cream with an increase of +7.5% **, +7.9% ** and +12% **** after 12 h, 7 days and 28 days, respectively (FIG. 5).

A significant increase in the antioxidant potential of the skin was also observed in the group treated with the “active” cream compared to the group treated with the placebo cream (reduction of Fe³⁺ to Fe²⁺ of +17% by day 28 compared to the placebo group, FIG. 6).

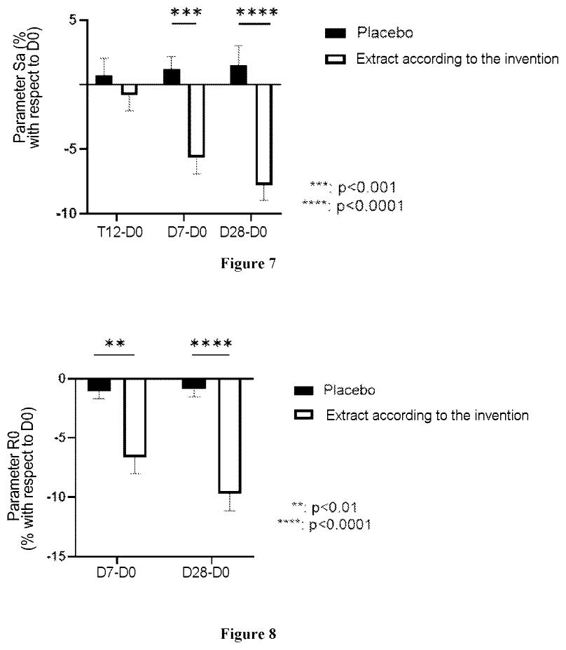

An improvement in the smooth appearance of the skin and in the biomechanical properties thereof was observed in the group treated with the “active” cream compared to the “placebo” group. Thus, the “active” cream comprising the extract according to the invention made it possible to smooth the skin and rapidly reduce the appearance of wrinkles, as evidenced by the decrease in the parameter Sa of −6.9% *** and −9% **** after 7 days and 28 days (FIG. 7). A clear reduction in crow's feet wrinkles can also be observed visually on the images taken on D0 and D28 and also confirmed by the measurements made by the Primos 3D.

The “active” cream also improved the biomechanical properties of the skin by increasing its firmness and elasticity, as illustrated by a significant decrease in the parameter R0 (FIG. 8) and a significant increase in the parameter R2 (FIG. 9) compared with the placebo group.

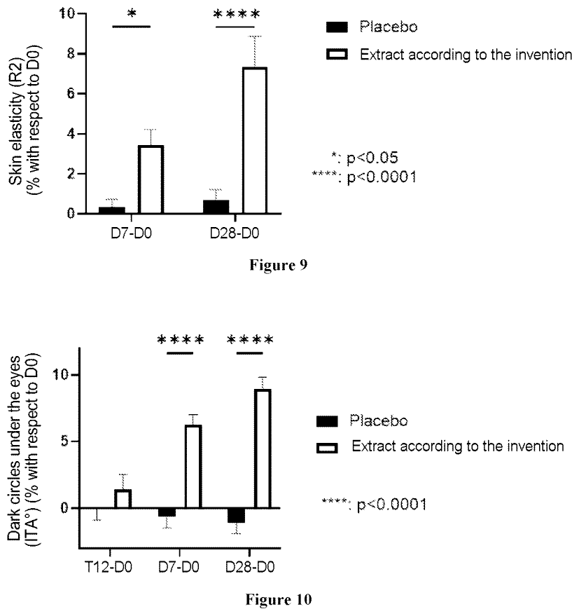

Finally, the “active” cream induced a rapid and significant lightening of the dark circles under the eyes compared to the placebo group (FIG. 10).

In conclusion, this clinical trial showed that the extract of Melaleuca alternifolia is a powerful antifatigue agent for the skin. The daily application of a cream comprising 0.0025% of the extract according to the invention made it possible to improve the signs of skin fatigue in people with disturbed sleep cycles.

The extract according to the invention notably made it possible to improve the barrier function of the skin, to improve the antioxidant power of the skin, to prevent and improve the smooth appearance and biomechanical properties of the skin and to reduce dark circles in volunteers, in a significant manner compared to the group of volunteers treated with placebo cream.

The scope of the present invention is not intended to be limited to the particular embodiments described, which are provided, for example, to illustrate various aspects of the invention. Various modifications or variants of the compositions and processes described will become obvious in light of the description and teachings provided in the present applications. Such modifications can be made without departing from the true scope and spirit of the present application and are intended to be included within the scope of the present patent application. Although the invention can be described in relation to specific preferred embodiments, it should be understood that the invention as claimed should not be unduly limited to such specific embodiments. Indeed, the various modifications or variants of the embodiments of the invention which are obvious to those skilled in the art in the cosmetic field or related fields are intended to fall within the scope of the following claims.

### Exemplary Embodiments

1. A cosmetic use of an oily extract of Melaleuca alternifolia, as a regenerative, repairing or anti-fatigue cosmetic agent for healthy skin, wherein said extract comprises:
   more than 20% of compounds selected from sesquiterpenes and sesquiterpenoids, and
   less than 6% of terpinen-4-ol, the percentages being expressed relative to the total content of volatile compounds present in the extract determined by gas chromatography coupled with a flame ionization detector (GC/FID).
2. The cosmetic use according to embodiment 1, characterized in that said extract of Melaleuca alternifolia comprises less than 15%, preferably less than 10%, better still less than 2.0% of terpineols.
3. The cosmetic use according to embodiment 1 or 2, characterized in that said extract of Melaleuca alternifolia comprises:
   less than 5.0%, preferably less than 2.0%, better still less than 1.0%, of terpinen-4-ol, and/or
   less than 5.0%, preferably less than 2.0%, better still less than 1.5%, of alpha-terpineol.
4. The cosmetic use according to any one of embodiments 1 to 3, characterized in that said extract of Melaleuca alternifolia comprises more than 30%, preferably more than 40% of compounds selected from sesquiterpenes and sesquiterpenoids.
5. The cosmetic use according to any one of embodiments 1 to 4, characterized in that said extract of Melaleuca alternifolia comprises:
   from 5% to 15% of aromadendrene,
   from 10% to 25% of ledene,
   from 10% to 25% of delta-cadinene,
   from 1% to 5% of globulol, and
   from 1% to 5% of viridifloral.
6. The cosmetic use according to any one of embodiment 1 to 5, characterized in that said extract of Melaleuca alternifolia is obtained by fractional distillation of an essential oil of leaves and/or terminal branches of Melaleuca alternifolia.
7. The cosmetic use according to any one of embodiments 1 to 6, wherein said extract of Melaleuca alternifolia is used to treat or prevent one or more signs of skin fatigue selected from a loss of radiance of the skin, a dull complexion, a peaky complexion, a loss of uniformity of the complexion, haggard facial features, dark circles around the eyes, bags under the eyes, puffy eyelids, an alteration in the smooth appearance of the skin, an increase in the roughness of the skin, a loss of elasticity, a loss of density, a loss of firmness, a loss of hydration, an appearance of wrinkles, an appearance of redness, and combinations thereof.
8. The cosmetic use according to any in one of embodiments 1 to 6, wherein said extract of Melaleuca alternifolia is used as a cosmetic agent for promoting or improving skin recovery during sleep and/or as a cosmetic agent for potentiating the natural mechanisms of skin repair and regeneration controlled by melatonin.
9. The cosmetic use according to any one of embodiments 1 to 8, wherein said extract of Melaleuca alternifolia is further used for increasing the total duration of sleep, and/or that of deep sleep.
10. The cosmetic use according to any one of embodiments 1 to 9, wherein said extract of Melaleuca alternifolia is present as a cosmetic active agent in a cosmetic composition, preferably a cream, a balm, a mask, a serum or a lotion.
11. An oily extract of Melaleuca alternifolia comprising:
   more than 20% of compounds selected from sesquiterpenes and sesquiterpenoids, and
   less than 6% of terpinen-4-ol, the percentages being expressed relative to the total content of volatile compounds present in the extract determined by gas chromatography coupled with a flame ionization detector (GC/FID).
12. The oily extract of Melaleuca alternifolia according to embodiment 11, characterized in that it is as defined in any one of claims 2 to 6.
13. A cosmetic ingredient or precursor composition for the preparation of a cosmetic composition comprising an extract of Melaleuca alternifolia of embodiment 11 or 12.
14. The cosmetic ingredient or precursor composition according to embodiment 13, comprising from 0.1% to 1.0% by weight, preferably from 0.1% to 0.5% by weight, of an extract of Melaleuca alternifolia of embodiment 11 or 12, in a cosmetically acceptable carrier.
15. The cosmetic ingredient or precursor composition according to embodiment 14, comprising or consisting essentially of 0.1% to 1.0% by weight of an extract of Melaleuca alternifolia of embodiment 11 or 12 in a cosmetically acceptable carrier selected from pentylene glycol or triheptanoin.
16. The cosmetic ingredient or precursor composition according to embodiments 13 to 15, wherein the extract of Melaleuca alternifolia extract is present as a regenerative, repairing or anti-fatigue active agent for healthy skin.
17. A cosmetic composition comprising the extract of Melaleuca alternifolia according to embodiments 11 or 12, or the cosmetic ingredient or precursor composition according to any one of embodiments 13 to 15, in combination with at least one cosmetically acceptable excipient.
18. The cosmetic composition according to embodiment 17, characterized in that it comprises from 0.0005% to 0.01%, preferably from 0.001% to 0.005% by weight of said extract of Melaleuca alternifolia.
19. The cosmetic composition according to embodiment 17, characterized in that it comprises from 0.5% to 5% by weight, preferably from 1% to 3% by weight, of said cosmetic ingredient.
20. The cosmetic composition according to any one of embodiments 17 to 19, said cosmetic composition being selected from the group consisting of aqueous solutions, aqueous-alcoholic solutions, oil-in-water (O/W) emulsions or water-in-oil (W/O) emulsions or multiple (triple: W/O/W or O/W/O) emulsions, nanoemulsions, in particular O/W nanoemulsions, aqueous gels, or spherule-assisted dispersions of a fatty phase in an aqueous phase, suspensions, preferably in aqueous or aqueous-alcoholic media, liposome suspensions, powders, lotions, milks, creams, unguents, gels, foams, and ointments.
21. The cosmetic composition according to any one of embodiments 16 to 20, said composition being a care product intended for use in the treatment or prevention of skin fatigue, for example a serum, a cream, a mask or a balm, preferably for use at night.
22. A cosmetic method for preventing, reducing or treating signs of skin fatigue in a subject, said method comprising the application of a cosmetically effective amount of an extract of Melaleuca alternifolia of embodiment 11 or 12, or of a cosmetic composition according to any one of embodiments 17 to 21 on the skin.
23. The use of an extract of Melaleuca alternifolia according to embodiment 11 or 12, or of a cosmetic ingredient according to any one of embodiments 13 to 15 in the manufacture of a cosmetic composition for treating, preventing or reducing the signs of skin fatigue.

1-23. (canceled)

24. An oily extract of Melaleuca alternifolia comprising:
more than 20% of compounds selected from sesquiterpenes and sesquiterpenoids, and less than 6% of terpinen-4-ol,
the percentages being expressed relative to the total content of volatile compounds present in the extract determined by gas chromatography coupled with a flame ionization detector (GC/FID).

25. The oily extract of Melaleuca alternifolia of claim 24, characterized in that it comprises less than 10% of terpineols.

26. The oily extract of Melaleuca alternifolia of claim 24, characterized in that said extract of Melaleuca alternifolia comprises:
less than 5.0% of terpinen-4-ol, and/or
less than 5.0% of alpha-terpineol.

27. The oily extract of Melaleuca alternifolia of claim 24, characterized in that it comprises more than 30%, of compounds selected from sesquiterpenes and sesquiterpenoids.

28. The oily extract of Melaleuca alternifolia of claim 24, characterized it comprises:
from 5% to 15% of aromadendrene,
from 10% to 25% of ledene,
from 10% to 25% of delta-cadinene,
from 1% to 5% of globulol, and
from 1% to 5% of viridifloral.

29. The oily extract of Melaleuca alternifolia of claim 24, which is obtained by fractional distillation of an essential oil of leaves and/or terminal branches of Melaleuca alternifolia.

30. A cosmetic ingredient comprising from 0.1% to 1.0% by weight of the oily extract of Melaleuca alternifolia of claim 24 in a cosmetically acceptable carrier.

31. The cosmetic ingredient of claim 30, wherein the cosmetically acceptable carrier is selected from pentylene glycol or triheptanoin.

32. A cosmetic composition comprising the extract of Melaleuca alternifolia of claim 24 in combination with at least one cosmetically acceptable excipient.

33. The cosmetic composition of claim 32, characterized in that it comprises from 0.0005% to 0.01 by weight of said extract of Melaleuca alternifolia.

34. The cosmetic composition of claim 32, characterized in that it is selected from the group consisting of aqueous solutions, aqueous-alcoholic solutions, oil-in-water (O/W) emulsions or water-in-oil (W/O) emulsions or multiple (triple: W/O/W or O/W/O) emulsions, nanoemulsions, aqueous gels, spherule-assisted dispersions of a fatty phase in an aqueous phase, suspensions, liposome suspensions, powders, lotions, milks, creams, unguents, foams, serums, balms, and ointments.

35. A cosmetic composition comprising the cosmetic ingredient of claim 30 in combination with at least one cosmetically acceptable excipient.

36. The cosmetic composition of claim 35, characterized in that it comprises from 0.5% to 5% by weight of the cosmetic ingredient.

37. A cosmetic method for preventing, reducing or treating one or more signs of skin fatigue in a subject, said method comprising the application of a cosmetically effective amount of an extract of Melaleuca alternifolia of claim 24 on the skin.

38. The cosmetic method of claim 37, wherein the one or more signs of skin fatigue are selected from a loss of radiance of the skin, a dull complexion, a peaky complexion, a loss of uniformity of the complexion, haggard facial features, dark circles around the eyes, bags under the eyes, puffy eyelids, an alteration in the smooth appearance of the skin, an increase in the roughness of the skin, a loss of elasticity, a loss of density, a loss of firmness, a loss of hydration, an appearance of wrinkles, an appearance of redness, and combinations thereof.

39. The cosmetic method of claim 37, wherein the extract of Melaleuca alternifolia is used as an active cosmetic agent for promoting or improving skin recovery during sleep and/or as an active cosmetic agent for potentiating the natural mechanisms of skin repair and regeneration controlled by melatonin.

40. The cosmetic method of claim 37, wherein the extract of Melaleuca alternifolia activates the signaling pathway of melatonin and/or potentiates the effect of melatonin on the skin.

41. The cosmetic method of claim 37, wherein the extract of Melaleuca alternifolia is applied on the skin in the form of a cosmetic composition.

42. The cosmetic method of claim 41, wherein the cosmetic composition is a serum, a cream, a mask or a balm.

43. The cosmetic method of claim 37, wherein the extract is applied on the skin before going to bed.
